(19) United States
(12) Patent Application Publication
(10) Pub. No.: US 2014/0323346 A1
(43) Pub. Date: Oct. 30, 2014

(54) BINDING AGENT

(71) Applicants: Garvan Institute of Medical Research, Darlinghurst, New South Wales (AU); Cancer Therapeutics CRC Pty Ltd., Bundoora, Victoria (AU)

(72) Inventors: Roger John Daly, Darlinghust (AU); Ian Peter Holmes, Bundoora (AU); Ian Street, Bundoora (AU); Scott Raymond Walker, Parkville (AU)

(73) Assignee: Garvan Institute of Medical Research, Darlinghurst, New South Wales (AU)

(21) Appl. No.: 14/359,577

(22) PCT Filed: Nov. 21, 2012
(86) PCT No.: PCT/AU2012/001434

§ 371 Date: May 20, 2014

Related U.S. Application Data
(60) Provisional application No. 61/561,969, filed on Nov. 21, 2011.

(51) Int. Cl. G01N 33/573

(52) U.S. Cl. CPC G01N 33/573
USPC 506/9; 544/295; 536/53; 506/15; 435/7.4; 435/6.13

(57) ABSTRACT

The present disclosure generally relates to protein binding agents, such as protein kinase binding agents of general Formula (I). The protein binding agents may be provided attached to a solid support and may be used, for example, to detect the presence of a broad range of proteins in a sample. Methods of synthesizing the protein binding agents, and kits comprising the protein binding agents, are also disclosed.

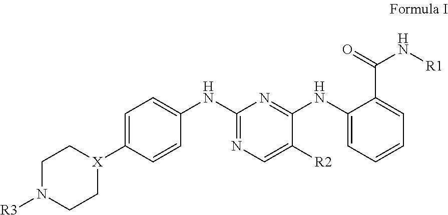

### FIELD OF THE INVENTION

The present disclosure generally relates to protein binding agents, to methods of their production and to the use of protein binding agents in isolating proteins. In one example, the present disclosure relates to protein kinase binding agents.

### BACKGROUND OF THE INVENTION

### Protein Kinases and Human Disease

The protein kinase complement of the human genome encompasses approximately 500 members, which can exhibit serine/threonine-, tyrosine-, or dual-specificity (Manning et al., 2002). A typical mammalian cell expresses ˜300 different protein kinases (Su et al., 2002). By phosphorylating specific protein targets, these enzymes play critical roles in mediating intracellular signalling events, and regulate diverse cellular processes, including proliferation, survival, metabolism and motility. In addition, protein kinases themselves are subject to intermolecular phosphorylation events that regulate enzyme activity and downstream signalling. For example, phosphorylation within the activation loop of the kinase domain stabilizes the catalytically active state of many kinases (Nolen et al., 2004), while autophosphorylation of receptor tyrosine kinases (RTKs) creates binding sites for specific effector molecules (Lemmon et al., 2010). However, despite intensive research on the protein kinase superfamily, many of its members remain largely uncharacterized in terms of both function and regulation.

Importantly, aberrant kinase signalling is strongly associated with many human diseases. For example, approximately one-third of protein kinase genes map to cancer amplicons (Manning et al., 2002), and many kinase genes are subject to oncogenic genomic rearrangements or mutations. For example, the tyrosine protein kinase Abl forms part of the Bcr-Abl fusion protein that drives the development of chronic myeloid leukemia (CML), and activating mutations in the serine/threonine protein kinase B-Raf and the receptor tyrosine kinase EGFR occur in approximately 40% of melanomas and 15-30% of non-small cell lung cancers (NSCLC), respectively. This has led to the development of effective therapies that selectively target the deregulated kinase, which include the small molecule tyrosine kinase inhibitors (TKIs) imatinib, erlotinib and PLX4032 for treatment of CML, EGFR-mutant NSCLC and B-Raf mutant melanoma, respectively (Knight et al., 2010; Brognard and Hunter, 2011; Pao and Chmielecki, 2010). In addition, particular protein kinases are implicated in other important human pathologies including inflammatory conditions (eg rheumatoid arthritis, inflammatory bowel disease) (Cohen, 2002), cardiovascular disease (Belmonte and Blaxall, 2011), neurological disorders and neurodegenerative disease (Su and Tsai, 2010), type II diabetes (Donath and Shoelson, 2011) and autosomal dominant polycystic kidney disease (Qin et al., 2010), and may represent diagnostic or prognostic markers, and/or therapeutic targets.

### Characterization of Cellular Signalling Networks by Mass Spectrometry

Recent advances in mass spectrometry (MS)-based proteomics allow global ‘snapshots’ to be taken of many types of post-translational modification, including protein phosphorylation, and thus provide the capability to comprehensively characterize cellular signalling networks (Macek et al., 2009). One example is the use of a combined immunoaffinity/MS approach to characterize phosphotyrosine signalling in particular cancer subtypes (Hochgrafe et al., 2010). However, this methodology does not detect the significant proportion of the human kinome that is not tyrosine-phosphorylated. In addition, due to the low cellular abundance of many protein kinases, peptide enrichment based solely on protein phosphorylation status leads to under-representation of the protein kinase subclass in subsequent MS-analyses, highlighting the need for an additional purification step (Daub et al., 2008). Recent studies have attempted to address this problem by the use of ATP-competitive small molecule kinase inhibitors as affinity reagents. For example, coupling of multiple broad-specificity kinase ligands to beads (to create linobeads') (Bantscheff et al., 2007), or use of a series of affinity columns containing inhibitors with distinct but overlapping selectivity profiles (Daub et al., 2008) has been performed in an attempt to isolate a broader range of protein kinases from cell extracts. When used in combination with quantitation techniques such as stable isotope labelling by amino acids in culture (SILAC), these approaches can be used to compare the expressed kinome, in terms of both protein levels and activation status, between different cell types and treatment conditions (Daub et al., 2008; Oppermann et al., 2009). However, the broad specificity kinase ligands previously described have only been shown to bind a limited subset of the total kinome. For example, seven different ATP-competitive inhibitors (Bis (III) indoyl-maleimide, purvalanol B, staurosporine, CZC8004 and the analogs of PD173955, sunitinib and vandetanib) bound to beads (“kinobeads”) only bound approximately 180 protein kinases from each of 5 different cell lines (Bantscheff et al., 2007); the kinase inhibitors VI16832, bisindoylmaleimide X, AX14596, SU6668 and purvalanol B used in a multicolumn affinity chromatography procedure (Daub et al., 2008) detected 219 protein kinases from HeLa cells; and of three different kinase capture reagents compared in Oppermann et al., 2009, the most effective was determined to be VI16832, which was shown to be able to detect 170 protein kinases in total from three different cell lines. There remains a need to identify kinase ligands with a still broader specificity in order to enable a more reliable analysis of the total kinase expression profile of a cell. In addition, kinase ligands are desired that would allow complex purification methods such as multicolumn affinity chromatography to be simplified.

### SUMMARY OF THE INVENTION

The present inventors have identified a number of particularly effective protein binding reagents. In particular, the inventors have identified a number of compounds that are particularly effective at binding a broad range of protein kinases. Accordingly, the present disclosure provides a compound of the following general Formula I:

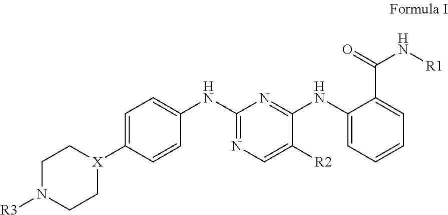

wherein R1=H or Me; R2=CF₃ or Cl; R3=H, COCH₃ or a linker group; and
X=N or C. Preferably, R1=H, R2=Cl, X=N and R3=H, COCH₃ or a linker group. In a particularly preferred embodiment, R1=H, R2=Cl, X=N and R3=H.

The present disclosure also provides methods of synthesizing the compound of Formula I, as described herein. Thus, the present disclosure provides a method of synthesizing the compound of Formula I, the method comprising reacting a compound of the following formula F4 (wherein R1=H or Me;

X=N or C; and the CF₃ group can optionally be substituted with Cl)

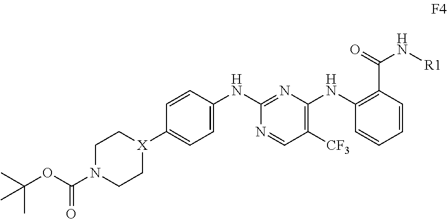

with an acid under suitable reaction conditions. In one example, the acid is trifluoroacetic acid.

In addition, the present disclosure provides a method of synthesizing the compound disclosed herein, the method comprising reacting an amino pyridine of the formula F6

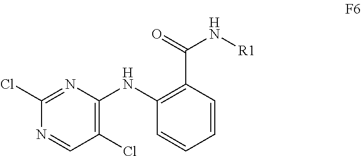

with an aniline of formula 11 or formula 12

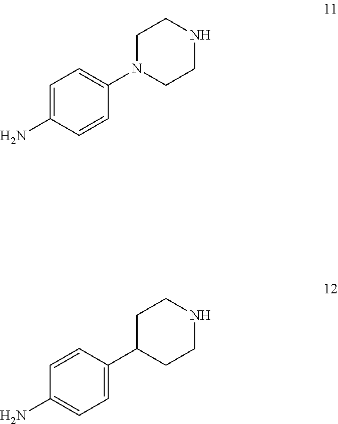 [SMILES: Nc1ccc(N2CCNCC2)cc1 ; Nc1ccc(C2CCNCC2)cc1]

under suitable reaction conditions.

The compound disclosed herein may be bound to a solid support. Thus, the present disclosure also provides a solid support having a compound disclosed herein attached thereto. In one example, the solid support may be any solid support capable of being used in a chromatography column. In a preferred example, the solid support comprises a plurality of sepharose beads.

The solid support may further comprise additional binding agents attached thereto. For example, the solid support may comprise one or more additional protein kinase binding agents attached thereto. In a preferred example, the solid support further comprises any one or more of bis (III) indoyl-maleimide, purvalanol B, staurosporine, CZC8004, sunitinib, vandetanib, VI16832, bisindoylmaleimide X, AX14596 and SU6668 (and most preferably, any one or more of purvalanol B, VI16832 and SU6668) attached thereto.

The present disclosure also provides a method of detecting the presence of one or more proteins in a sample, the method comprising contacting the sample with the compound disclosed herein or the solid support disclosed herein. Preferably, the one or more proteins is a protein kinase, since the compound of Formula I has been shown to be particularly effective as a protein kinase capture reagent. In one example, the compound of Formula I has been shown to be particularly effective at binding to protein kinases of the STE (homologues of yeast sterile 7, sterile 11 and sterile 20), CMGC (containing cyclin-dependent kinase, mitogen-activated protein kinase, glycogen synthase kinase 3 and CDC2-like) and AGC (containing protein kinase A, G and C) subfamily, and to protein kinases of the Akt family (also known as the Protein Kinase B family).

The methods disclosed herein may be performed on a sample taken from a subject. The sample may comprise cells, which may be lysed or solubilized (for example, by contacting the cells with a detergent) before the sample is contacted with the compound disclosed herein or the solid support disclosed herein. In one example, the sample is taken from a subject suffering from or suspected of suffering from a disease. Thus, the methods disclosed herein can be used to determine the protein kinase expression profile in a subject suffering from a disease. The type of disease is not limiting on the application of the methods disclosed herein. Thus, the sample can be taken from a subject suffering from or suspected of suffering from any disease. In one example, the disease is cancer. In another example, the disease is an inflammatory condition (for example, rheumatoid arthritis, inflammatory bowel disease, or another inflammatory condition), a cardiovascular disease, a neurological disorder or neurodegenerative disease, type II diabetes or autosomal dominant polycystic kidney disease.

The methods disclosed herein may be used to isolate one or more proteins from a sample. Thus, in one example, the methods are useful in isolating and/or purifying one or more protein kinases from a sample. The isolation of one or more protein kinases from a sample may be particularly advantageous in diagnostic or prognostic methods relying on the detection of the presence or level of expression of one or more protein kinases in a sample.

The present disclosure also provides a method of diagnosing the presence of a disease or a predisposition to a disease in a subject, the method comprising:

detecting the presence of one or more proteins in a sample taken from the subject by contacting the sample with the compound or solid support disclosed herein,

wherein the presence of the one or more proteins in the sample is indicative of the disease or predisposition thereto.

The present disclosure also provides a method of monitoring a subject's response to a therapeutic treatment for a disease, the method comprising:

detecting the presence of one or more proteins in a sample taken from the subject by contacting the sample with the compound or solid support disclosed herein at a first time point; and

detecting the presence of one or more proteins in a sample taken from the subject by contacting the sample with the compound or solid support disclosed herein at a second, later time point after the subject has been exposed to a therapeutic treatment,

wherein the presence of the one or more proteins in the sample at the second time point is indicative of the subject's response to the therapeutic treatment.

The present disclosure also provides a method of screening for an agent capable of binding a protein kinase, the method comprising contacting the binding agent or solid support disclosed herein with a sample comprising one or more protein kinases in the presence and in the absence of a test agent, and identifying the test agent as an agent capable of binding a protein kinase if the level of binding of the binding agent or the solid support to any one or more of the protein kinases present in the sample is reduced in the presence, compared to the absence of the test agent. Thus, the present disclosure provides competition binding assays that can be used, for example, to investigate the binding affinity of a test agent to a broad range of protein kinases.

In addition, the present disclosure provides a kit comprising the compound and/or the solid support as disclosed herein, and instructions for use.

The features of any embodiment described herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The invention is hereinafter described by way of the following non-limiting Examples and with reference to the accompanying figures.

### BRIEF DESCRIPTION OF THE ACCOMPANYING DRAWINGS

FIG. 1. Workflow for affinity purification of kinases from cellular lysates using kinase capture reagents. “CTx compound” refers to any compound of the general Formula I, wherein R1=H or Me; R2=CF₃ or Cl; R3=H, COCH₃ or a linker group; and X=N or C.

FIG. 2. Characterization of proteins bound by CTx-0294885.

A. Distribution of bound kinases amongst the different protein kinase families. Cell lysates from MDA-MB-231 breast cancer cells were subject to affinity purification on a CTx-0294885 affinity column and bound kinases were identified by LC-MS/MS. The pie-chart indicates the number of kinases in each family that were identified (AGC=containing protein kinase A, G and C; CAMK=calcium/calmodulin-dependent protein kinase; CMGC=containing cyclin-dependent kinase, mitogen-activated protein kinase, glycogen synthase kinase 3 and CDC2-like; TK=tyrosine kinase; TKL=tyrosine kinase-like; STE=homologues of yeast sterile 7, sterile 11 and sterile 20; CK1=casein kinase 1). No kinases from the minor RGC (receptor guanylate cyclase) family were identified.

B. Relative enrichment of different kinase families by the CTx-0294885 affinity resin. The histograms indicate the representation of a given kinase family within the CTx-0294885-bound fraction (“CTx-0294885”, left bar of pairs, calculated as the number of CTx-0294885-bound kinases within a given kinase family/total number of kinases bound by CTx-0294885) and the representation of each kinase family within the total kinome (“All kinases”, right bar of pairs, calculated as the number of protein kinases in each family/total number of human protein kinases).

C. Gene ontology classification of non-protein kinases bound by CTx-0294885. Non-protein kinases bound by CTx-0249885 were compared with the entire list of UniProtKB entries. Over-represented Gene Ontology (GO) molecular function terms with statistical significance of p<0.001 were identified and of these, only the top 30 GO terms with the lowest p value are shown. Fold change for each GO term was calculated by dividing the CTx-0249885-bound ratio (ratio of CTx-0249885-bound proteins annotated to a particular GO term/total number of CTx-02498850-bound proteins) by the total ratio (total number of proteins annotated to that particular GO term/total number of proteins in the database).

FIG. 3. Comparison of the binding selectivity of CTx-0294885 with other commonly-used kinase capture reagents. Cell lysates from MDA-MB-231 breast cancer cells were subject to affinity purification on columns containing purvalanol B (P), SU6668 (S), VI16832 (V) or CTx-0294885. The Venn diagram indicates the total number of kinases bound by the P, S and V affinity columns (P/S/V) and the CTx-0294885 column, as well as overlap between the 2 groups.

FIG. 4. Use of CTx-0294885 in combination with other commonly-used kinase capture reagents. A. Combining CTx-0294885 with other kinase capture reagents greatly enhances kinome coverage. Cell lysates from MDA-MB-231 breast cancer cells were subject to affinity purification on columns containing a mixture of P, S and V (Mix 3) or P, S, V and CTx-0294885 (Mix 4). The Venn diagram indicates the total number of kinases bound by Mix 3 and Mix 4, as well as overlap between the 2 groups. B. Additional kinases in each family identified by Mix 4 compared with Mix 3.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−20%, more preferably +/−10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the terms “treating”, “treat” or “treatment” include administering a therapeutically effective amount of an agent sufficient to reduce or eliminate at least one symptom of disease.

As used herein, the term “diagnosis”, and variants thereof, such as, but not limited to “diagnose” or “diagnosing” shall include, but not be limited to, a primary diagnosis of a clinical state or any primary diagnosis of a clinical state or a primary diagnosis of a predisposition to developing a clinical state. The diagnostic methods disclosed herein are also useful for monitoring disease progression, or for monitoring a subject's response to therapy, or for monitoring disease recurrence. For example, in the case of cancer, the methods disclosed herein are useful for assessing the remission of a subject, or for monitoring tumour recurrence, such as following surgery, radiation therapy, adjuvant therapy or chemotherapy, or for determining the appearance of metastases of a primary tumour. All such uses of the assays described herein are encompassed by the present disclosure.

As used herein, the term “subject” refers to an animal, (e.g., a mammal) or a plant (e.g., any monocotyledonous or dicotyledonous plant). In a preferred embodiment, the subject is mammalian, for example a human. Other preferred embodiments include livestock animals such as horses, cattle, sheep and goats, as well as companion animals such as cats and dogs. In another preferred embodiment, the subject is an insect. The insect may be a known vector of an infectious disease. In one example, the insect is a mosquito, (for example, of the genus Anopheles, such as Anopheles gambiae, Anopheles arabiensis, Anopheles merus, Anopheles melas, Anopheles atroparvus, or other species). As will be appreciated by a person skilled in the art, the compound disclosed herein can be used to identify potential therapeutic targets in any animals which cause or contribute to the spread of disease. In another preferred embodiment, the subject is a plant which is a crop plant (for example, cereals and pulses, maize, wheat, potatoes, tapioca, rice, sorghum, millet, cassaya, barley, or pea), or other legume.

As used herein, the terms “conjugate”, “conjugated”, “link”, “linked”, “bind”, “bound”, “attach”, “attached”, or variations thereof are used broadly to refer to any form of covalent or non-covalent association between a compound disclosed herein and another agent.

### Protein Binding Agent

The present disclosure describes, for the first time, a compound of the following general Formula I. Such compounds can be used as protein binding agents. The compounds are capable of binding a broad range of proteins, including protein kinases and other purine nucleotide binding proteins. The compounds are particularly useful as protein kinase binding agents. In this regard, the compounds have been shown to bind a particularly broad range of protein kinases. The compounds disclosed herein can therefore be used in any application involving the detection of proteins (such as protein kinases) in a sample and/or the isolation of proteins (such as protein kinases) from a sample.

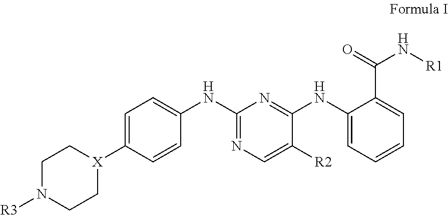

wherein R1=H or Me; R2=CF₃ or Cl; R3=H, COCH₃ or a linker group; and X=N or C.

In a preferred embodiment, the present disclosure provides a compound of Formula I, wherein R1=H, R2=Cl, X=N and R3=H, COCH₃ or a linker group. In a particularly preferred embodiment, the present disclosure provides a compound of Formula I, wherein R1=H, R2=Cl, X=N and R3=H. These compounds, whether provided alone or attached via a linker to a solid support, have proven to be particularly effective as protein binding agents.

The linker group may be any linker group capable of covalently attaching the compound to a solid support. The linker group may be of any size. The size of the linker group may be selected so as to reduce the chances of the solid support interfering with the binding of the compound of Formula I to a protein, such as a protein kinase. In one example, the linker group is between 2 and 18 atoms long. Thus, the linker group may be any of 2, 3, 4, 5, 6, 7, 8, 9, 10, 11, 12, 13, 14, 15, 16, 17 or 18 atoms long. In certain embodiments, the linker group may be composed exclusively of carbon atoms, or may contain both carbon and heteroatoms (for example oxygen, nitrogen or sulphur). The linker group may comprise a straight or branched hydrocarbon chain. The linker group may comprise one or more groups that increase the hydrophilicity of the linker. Thus, the linker group may comprise one or more hydrophilic groups. Suitable hydrophilic groups are known in the art and include, without limitation, polyethylene glycol (PEG) groups, alcohols, and others. The straight or branched hydrocarbon chain may comprise one or more heteroatoms within the chain, or branched from the chain. For example, the linker group may comprise a hydrocarbon chain comprising one or more PEG groups present within the chain. Alternatively or additionally, the linker group may comprise an alcohol (such as a secondary alcohol) branched from a hydrocarbon chain. Other alternatives will be apparent to a person skilled in the art. In certain embodiments, the linker group may comprise one or more carbonyl and/or carboxylic acid groups. The linker group may also comprise one or more imidate and/or imine groups.

The linker group may be functionalised. Suitable groups for functionalisation of the linker include, but are not limited to, activated esters (for example N-hydroxy succinate esters or pentafluorophenol esters), mixed anhydrides, acid chlorides, epoxides or isocyanates. Alternatively, linkers bearing carboxylic acids may be coupled in the presence of suitable coupling agents, for example, any one or more of HATU (O-(7-Azabenzotriazol-1-yl)-N,N,N′,N′-tetramethyluronium hexafluorophosphate; also known as 2-(1H-7-Azabenzotriazol-1-yl)-1,1,3,3-tetramethyl uronium hexafluorophosphate Methanaminium), EDCI (1-ethyl-3-(3-dimethylaminopropyl)carbodiimide), N-(3-dimethylaminopropyl)-N-ethylcarbodiimide hydrochloride (EDC), and others.

In a preferred embodiment, the linker group comprises an aminocarboxylic acid, (such as aminocaproic acid). The linker group may be attached to the solid support with cyanogen bromide or epichlorohydrin (preferably, cyanogen bromide). Thus, the solid support may comprise sepharose with aminocaproic acid attached thereto by CNBr activation (“CH sepharose 4B”), or sepharose with aminocaproic acid attached thereto by epichlorohydrin activation (“ECH sepharose 4B”). Alternative aminocarboxylic acids having a different length to aminocaproic acid may be used. In addition, alternative sepharose solid supports may be used, which have been alkylated with epichlorohydrin in order to enable attachment of a linker group thereto. In another embodiment, the linker group may be attached to the solid support via a coupling agent such as a carbodiimide, such as N-(3-dimethylaminopropyl)-N-ethylcarbodiimide hydrochloride (EDC).

In addition, the linker group may be cleavable. Thus, the linker may be capable of being cleaved or removed from the compound disclosed herein.

### Synthesis Methods

The compounds disclosed herein can be prepared, for example, by employing the following general methods. In addition, the compounds disclosed herein can be prepared, by way of a more specific example, using the procedures described in detail in the Examples. The reaction conditions referred to in the general and specific methods described herein are illustrative and non-limiting.

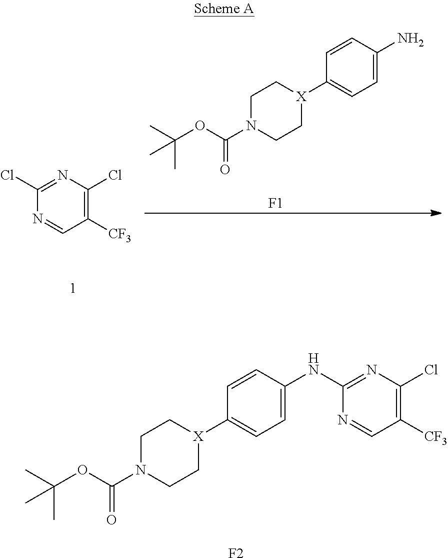

Commercially available 2,4-dichloro-5-(trifluoromethyl)pyrimidine (1) may be reacted with substituted synthetic anilines of formula F1, wherein X=N or C (as prepared, for example, using methods described in scheme B and C) under suitable reaction conditions to form intermediates of formula F2, wherein X=N or C. Thus, the present disclosure provides a compound of formula F2, wherein X=N or C. This compound is useful as an intermediate in the production of the compound of Formula I. The present disclosure also provides a method of synthesizing the compound of formula F2 wherein X=N or C, the method comprising reacting 2,4-dichloro-5-(trifluoromethyl)pyrimidine with one or more substituted synthetic anilines of formula F1 (wherein X=N or C) under suitable reaction conditions. The suitable reaction conditions may be determined by a person skilled in the art, and can include the selection of an appropriate solvent, reaction temperature, the addition of a Lewis acid (for example ZnCl₂ in diethyl ether), and other conditions. Regiochemical mixtures and di-substitution products may be obtained and regioisomers may be separated by known methods, such as chromatography.

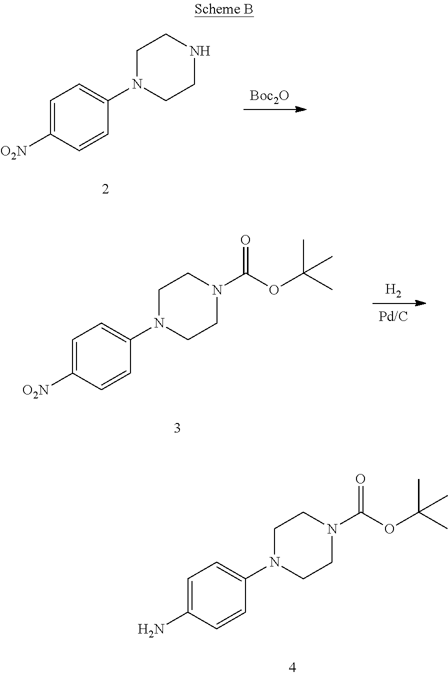 [SMILES: "Boc2O" ; O=[N+]([O-])c1ccc(N2CCNCC2)cc1 ; CC(C)(C)OC(=O)N1CCN(c2ccc([N+](=O)[O-])cc2)CC1 ; "H2" ; CC(C)(C)OC(=O)N1CCN(c2ccc(N)cc2)CC1]

Commercially available 1-(4-nitrophenyl)piperazine (2), or a salt thereof, can be reacted with Boc anhydride (C₁₀H₁₈O₅) to give tert-butyl 4-(4-nitrophenyl)piperazine-1-carboxylate (3). Subsequent reduction via hydrogenation in the presence of a catalyst, for example palladium on charcoal, gives the corresponding aniline, tert-butyl 4-(4-aminophenyl)piperazine-1-carboxylate (4). Alternative catalysts may be used.

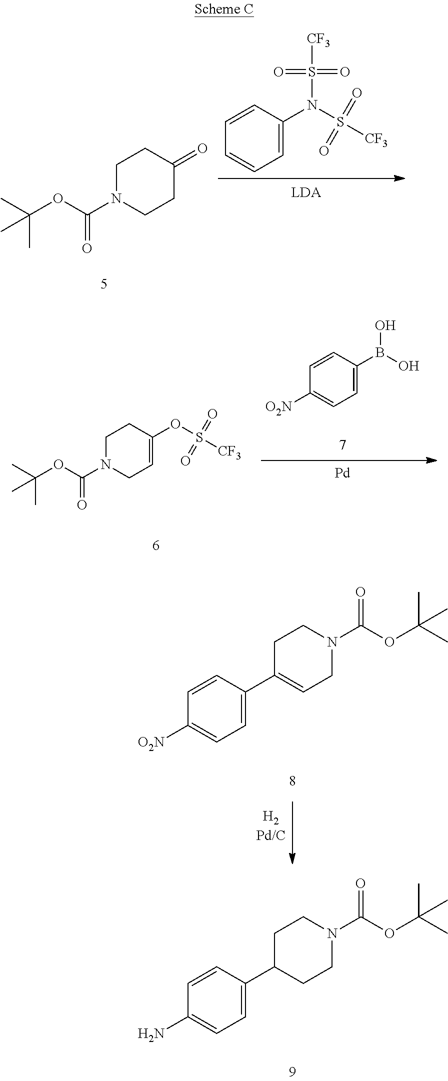

The corresponding 4-piperidine analogue of (4) can be prepared by a sequence of reactions starting with the conversion of commercially available tert-butyl 4-oxopiperidine-1-carboxylate (5) to vinyl triflate (tert-butyl 4-(((trifluoromethyl)sulfonyl)oxy)-5,6-dihydropyridine-1 (2H)-carboxylate) (6) by reaction with, e.g., phenyl triflimide (N,N-Bis(trifluoromethylsulfonyl)aniline). Coupling of (6) in a Suzuki type reaction (i.e., an organic reaction of an aryl- or vinyl-boronic acid with an aryl- or vinyl-halide catalyzed, for example, by a palladium complex) with (4-nitrophenyl)boronic acid (7) gives tetrahydropyridine (8). Subsequent reduction via hydrogenation in the presence of a catalyst, for example palladium on charcoal, gives anilino-piperidine (9).

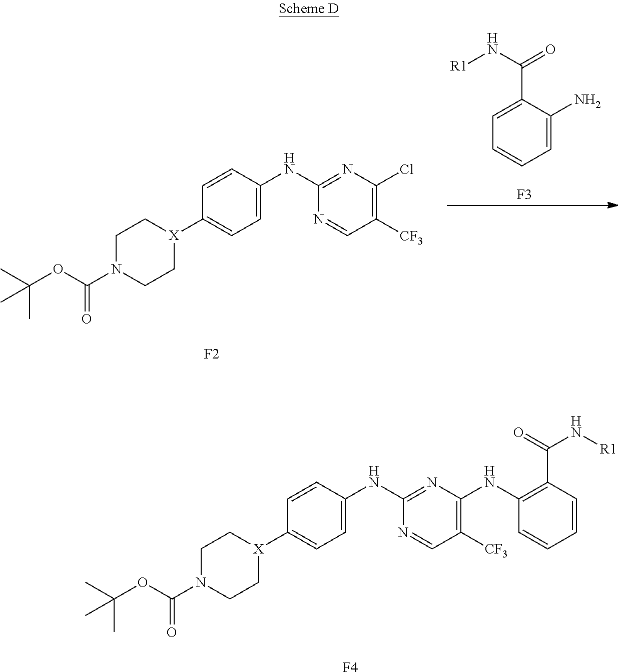

Chlorides of the formula F2 (wherein X=N or C) may be substituted with commercially available anilines of the formula F3 (where R1=H or Me) to give di-amino pyrimidines of the formula F4 (where R1=H or Me) by heating in the presence of a tertiary amine, for example diisopropylethylamine.

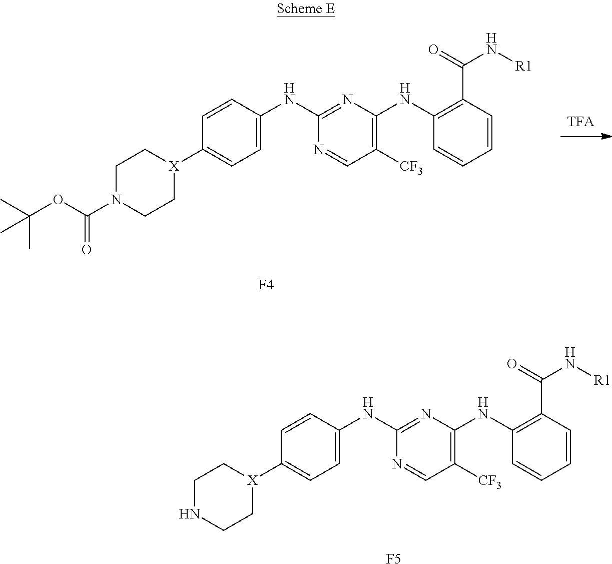

Di-amino-pyrimidines of the formula F4 may then be BOC deprotected by treatment with a suitable acid, for example trifluoroacetic acid, to give amines of the formula F5.

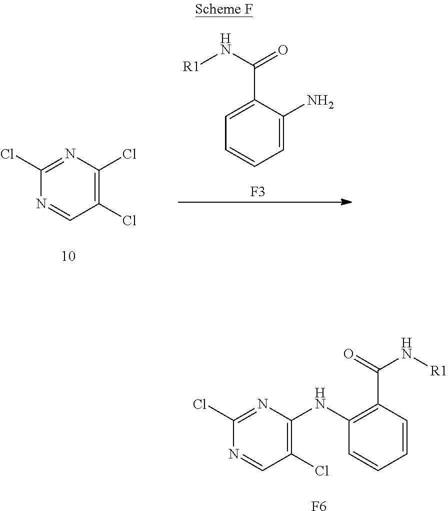

Commercially available 2,4,5-trichloro-pyrimidine (10) may be reacted with anilines of the formula F3, by heating in the presence of a tertiary amine, for example diisopropylethylamine, to give 4-amino pyrimidines of the formula F6. Where regiochemical mixtures and di-substitution are obtained the regioisomers may be separated by known methods, such as chromatography.

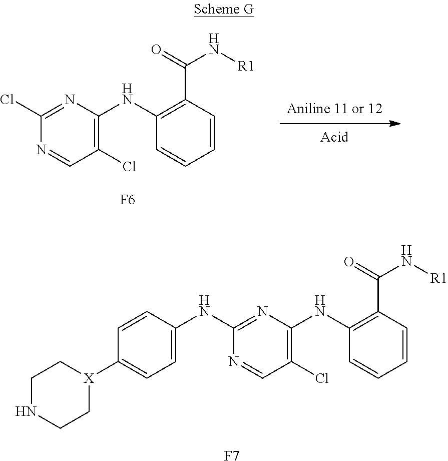

4-Amino pyrimidines of the formula F6 can be reacted with anilines (11) or (12) (as prepared, for example, using methods described in scheme H and I) in the presence of a suitable acid and solvent, for example hydrochloric acid in trifluoroethanol, to give amines of the formula F7 (which fall within the scope of Formula I).

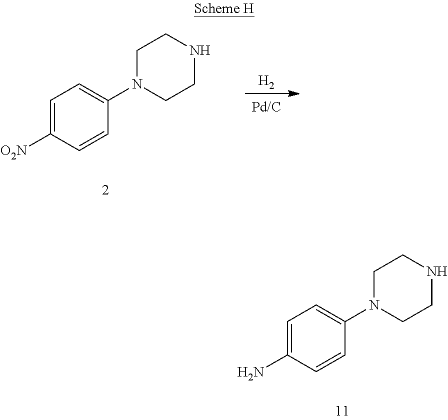 [SMILES: O=[N+]([O-])c1ccc(N2CCNCC2)cc1 ; "H2" ; Nc1ccc(N2CCNCC2)cc1]

Commercially available 1-(4-nitrophenyl)piperazine (2), or a salt thereof, can be reduced via hydrogenation in the presence of a catalyst, for example palladium on charcoal, to give the corresponding aniline, 4-(piperazin-1-yl)aniline (11).

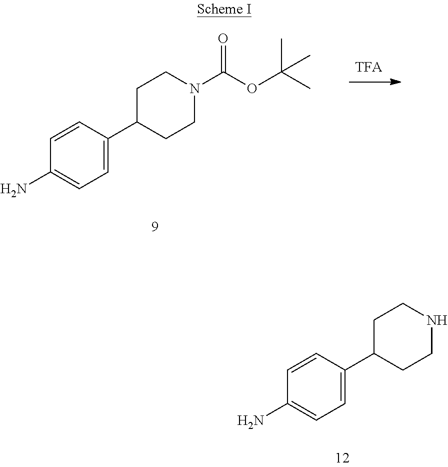 [SMILES: CC(C)(C)OC(=O)N1CCC(c2ccc(N)cc2)CC1 ; Nc1ccc(C2CCNCC2)cc1]

Anilino piperidine 9, prepared as described above, can be BOC-deprotected in the presence of a suitable acid, for example trifluoroacetic acid (TFA), to give 4-(piperidin-4-yl)aniline (12).

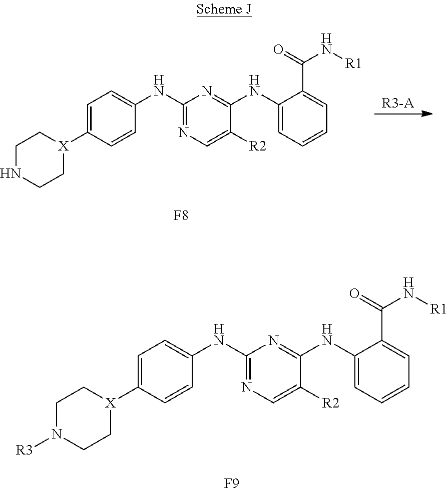

Amines of the formula F8 (wherein X=N or C; R1=H or Me; R2=CF₃ or Cl) may be reacted with solid supports (for example resins), bearing suitably functionalised linker chains, to give support bound compounds of the formula F9 (wherein X=N or C; R1=H or Me; R2=CF₃ or Cl; and R3=a linker group).

### Solid Supports

The compounds disclosed herein may be bound to a solid support. Thus, the present disclosure also provides any one or more of the compounds disclosed herein, bound to a solid support. The solid support may be any support capable of immobilising the compound in a chromatography column. Thus, the solid support may be any support capable of forming the stationary phase in a chromatography column.

In one example, the solid support is a resin, such as an agarose resin, a sepharose resin, or a mixed agarose/sepharose resin. In one example, the sepharose resin is a CH-sepharose 4-B resin. The resin may be provided in the form of one or more beads. In a preferred embodiment, the solid support comprises sepharose beads.

The solid support may be activated in order to facilitate binding of any of the compounds disclosed herein to the support. Suitable activation chemistries are known in the art, and include, for example, cyanogen bromide (CNBr) activation and reductive amination of aldehydes to attach proteins to a solid support such as an agarose and/or sepharose resin through lysine side chains. Other means of activating a solid support in order to facilitate binding of any of the compounds disclosed herein to the support will be apparent to a person skilled in the art.

Thus, the present disclosure provides a solid support having a compound as disclosed herein bound thereto. The solid support may comprise additional protein binding agents bound thereto. For example, the solid support may comprise one or more additional protein kinase binding agents bound thereto. Suitable additional protein kinase binding agents are known in the art and include (but are not limited to) Bis (III) indoyl-maleimide, purvalanol B, staurosporine, CZC8004 and the analogs of PD173955, sunitinib and vandetanib (Bantscheff et al., 2007), VI16832, bisindoylmaleimide X, AX14596, SU6668 (Daub et al., 2008), and others. Thus, the solid support disclosed herein may comprise the compound of Formula I and any one or more of Bis (III) indoyl-maleimide, purvalanol B, staurosporine, CZC8004 and the analogs of PD173955, sunitinib and vandetanib, VI16832, bisindoylmaleimide X, AX14596 and SU6668 bound thereto. In a preferred example, the solid support disclosed herein comprises a compound of Formula I and any one or more of purvalanol B, SU6668 and VI16832 bound thereto. In a particularly preferred embodiment, the solid support comprises a compound of Formula I and purvalanol B, SU6668 and VI16832 bound thereto.

The particular localization of each of the binding agents on the solid support may vary. Thus, each of the binding agents may be immobilised at a particular zone on the solid support so that a sample is contacted with one binding agent in one zone before contacting another binding agent in another zone. Alternatively, the binding agents may be randomly immobilised on the solid support. Where the solid support comprises one or more beads, each bead may have a particular binding agent attached thereto, or a mixture of binding agents attached thereto. Thus, the solid support may be provided as a mixture of beads, each bead having a different binding agent attached thereto, or as a mixture of beads, each bead having a mixture of binding agents attached thereto.

The binding agent disclosed herein may be attached to any proportion of the solid support. For example, when the solid support comprises a plurality of beads, any proportion of the beads may be provided with the compound of Formula I attached thereto. For example, the present disclosure provides a solid support comprising a plurality of beads (such as sepharose and/or agarose beads), wherein at least 5%, at least 10%, at least 20%, at least 30%, at least 40%, at least 50%, at least 60%, at least 70%, at least 80%, at least 90%, at least 95%, or at least 99% of the plurality of beads have a compound of Formula I attached thereto.

The solid support is preferably suitable for use in a chromatography column. Thus, the present disclosure also provides a chromatography column comprising a compound and/or a solid support as disclosed herein.

The present disclosure also provides a method of making a solid support having a protein binding agent attached thereto, the method comprising attaching a compound of Formula I to the solid support disclosed herein. As will be appreciated, the method of attaching a compound of Formula I to the solid support may vary depending on the linker and/or activation chemistry applied. In one example, the compound disclosed herein is attached to the solid support in a carbodiimide-mediated reaction. The carbodiimide may be N-(3-dimethylaminopropyl)-N-ethylcarbodiimide hydrochloride (EDC). The reaction may be carried out in the presence of a coupling buffer.

### Applications of the Binding Agent

The compound disclosed herein can be used to detect the presence of one or more proteins in a sample. In particular, the compound can be used to detect the presence of one or more protein kinases in a sample. Thus, the present disclosure provides a method of detecting the presence of one or more proteins in a sample, the method comprising contacting the sample with the compound disclosed herein or a solid support disclosed herein having the compound attached thereto. Due to the broad specificity of the compound for a range of protein kinases, the compound is particularly suited for use in determining the kinase expression profile in a sample taken from a subject.

In addition, the methods may be employed to identify potential therapeutic targets in a host. For example, determining the protein expression profile (for example, the protein kinase expression profile) in a sample taken from a subject known to suffer from a particular disease, and comparing that expression profile with the expression profile in a sample taken from a healthy subject, or from healthy tissue in the same subject, may identify an increased or decreased level of one or more proteins in the disease sample. Such proteins may represent potential therapeutic targets for treating or preventing the disease, or may represent diagnostic and/or prognostic markers of the disease.

The methods disclosed herein may also be used to investigate the effect of a particular agent (such as a known or potential therapeutic agent) on the protein expression profile (for example, a protein kinase expression profile) in a subject. Such methods may therefore be used to monitor the effect of a known or potential therapeutic agent on the expression of one or more protein kinases in a subject.

In addition to its use in detecting levels of proteins (such as protein kinases) in a sample, the compound disclosed herein can be used to identify certain molecular features of a range of proteins in a sample, such as the phosphorylation pattern, methylation pattern, acetylation pattern, and/or other molecular modification patterns of a broad range of protein kinases in a sample. Such additional steps of determining certain molecular features of the proteins to which the compound binds, (for example, determining the phosphorylation, methylation and/or acetylation pattern of protein kinases) may be comprised in the methods disclosed herein. The determination of phosphorylation pattern may comprise determining the phosphorylation state (i.e., determining the presence, absence, number and/or location of one or more phosphate groups) of the proteins to which the compound binds. The phosphorylation state may define the activation state of one or more protein kinases in a sample. The determination of methylation pattern may comprise determining the methylation state (i.e., determining the presence, absence, number and/or location of one or more methyl groups) of the proteins to which the compound binds. The methylation state may define the activation state of one or more protein kinases in a sample. The determination of acetylation pattern may comprise determining the acetylation state (i.e., determining the presence, absence, number and/or location of one or more acetyl groups) of the proteins to which the compound binds. The acetylation state may define the activation state of one or more protein kinases in a sample. Such methods have a wide range of experimental applications, including (but not limited to) determining the effect a particular protein-binding therapeutic agent has on the phosphorylation of a broad range of protein kinases in a subject, determining the phosphorylation state of expressed kinases in a disease state.

As disclosed herein, the compound is capable of detecting a high proportion of all kinases expressed in a host cell. For example, the compound disclosed herein is capable of detecting at least 150, at least 180, at least 190, at least 200, at least 210, at least 220, at least 230, at least 240, at least 250, at least 260, at least 270, or at least 280 different protein kinases in a cell sample. In addition, the compound disclosed herein has been shown to be particularly effective at binding a high number of protein kinases in the STE (homologues of yeast sterile 7, sterile 11 and sterile 20), CMGC (containing cyclin-dependent kinase, mitogen-activated protein kinase, glycogen synthase kinase 3 and CDC2-like) and AGC (containing protein kinase A, G and C) subfamilies of protein kinases. In one example, the compound disclosed herein has been shown to be particularly effective at binding all currently known members of the Akt family (Akt 1, Akt2 and Akt3 (Toker and Yoeli-Lerner, 2006; Zdychova and Komers, 2005)). Thus, in one example, the compound disclosed herein can be used to detect the presence of one or more, or all members of the Akt family. Accordingly, the compound disclosed herein can be used to study Akt-associated signalling networks.

The methods disclosed herein may be performed on any sample that may be taken from a subject. In one example, the sample is taken from a mammalian subject such as a human subject, may comprise a cell sample, tissue sample, or bodily fluid sample. The sample may originate from any number of sources, including for example (but not limited to) tissue biopsy, tumour, lymph node tissue, blood, or other source. The sample may be taken from a local disease site in a subject, such as a tumour. The sample may be removed from a subject by any suitable method known in the art. In one embodiment, the sample comprises a breast cancer cell, such as a cell of the cell line MDA MB 231. In another embodiment, the sample comprises a prostate cancer cell and/or a muscle cell and/or an adipose cell.

The sample may be subjected to any pre-treatment steps required to make any proteins in the sample accessible to the compound disclosed herein. Thus, where the sample is a cell sample, the cells may be lysed or solubilized before the sample is contacted with the compound disclosed herein. In one example, the lysis/solubilizing is performed using a detergent-based buffer. Suitable buffers are known in the art. Additional, optional pre-treatment steps such as partial purification steps will be apparent to a person skilled in the art. However, due to the high affinity exhibited by the compound herein for a wide range of protein kinases, the requirement for pre-treatment of the sample is minimal. For example, initial purification/filtering steps can be performed, but may not be required.

Once the compound or solid support disclosed herein has been contacted with the sample, the proteins bound to the compound or solid support may be obtained for further investigation by elution.

The methods disclosed herein may further comprise a step of identifying specific proteins (such as specific protein kinases) which bind to the compound disclosed herein. Any suitable identification methods may be used. Preferably, the methods comprise a step of identifying specific proteins (such as specific protein kinases) which bind to the compound using mass spectrometry. Alternative methods such as enzyme linked immunosorbent assay (ELISA) assays and/or western blot analysis may alternatively or additionally be used. For example, an ELISA assay can be performed to identify one or more particular target proteins in the sample. The one or more particular target proteins may be indicative of a particular disease state or may have prognostic value. For example, an ELISA assay may be performed in order to detect the presence and/or level of expression of a particular set of protein kinases which have been determined to be indicative of a subject's susceptibility to disease. Antibody arrays have been described, which can be used to detect such a kinase expression signature, and such arrays can also be used in the methods disclosed herein. In addition, individual kinases (for example, HER2) may specifically be analysed by these additional methods.

The methods disclosed herein may comprise the use of stable isotope labelling with amino acids in cell culture (SILAC) in order to quantify the amount of proteins bound to the compound disclosed herein. SILAC relies on metabolic incorporation of a given ‘light’ or ‘heavy’ form of the amino acid into the proteins. The method relies on the incorporation of amino acids with substituted stable isotopic nuclei (e.g. deuterium, 13C, 15N). Thus in an experiment, two cell populations are grown in culture media that are identical except that one of them contains a ‘light’ and the other a ‘heavy’ form of a particular amino acid (e.g. 12C and 13C labeled L-lysine, respectively). When the labeled analog of an amino acid is supplied to cells in culture instead of the natural amino acid, it is incorporated into all newly synthesized proteins. After a number of cell divisions, each instance of this particular amino acid will be replaced by its isotope labeled analog. The proportions of labelled amino acids can be quantified during mass spectrometry, thereby identifying which of the two cell populations each protein is derived from. Thus, the samples used in the methods disclosed herein may be cultured in the presence of one or more labelled amino acids before the sample is contacted with the binding agent disclosed herein.

Additional kinase treatment steps such as gel electrophoresis and/or protein digestion can also be performed in the methods disclosed herein, during the analysis of proteins bound to the compound or solid support disclosed herein.

As will be appreciated, the compound disclosed herein can be used to isolate one or more proteins (such as protein kinases) from a sample. Thus, the methods disclosed herein can be used in any methods where the isolation, purification and/or removal of protein kinases from a sample is desired. Thus, the methods disclosed herein provide the isolation of one or more proteins (such as protein kinases) from a sample.

The isolation of one or more proteins (such as protein kinases) from a sample may be useful as a step in a diagnostic and/or prognostic test. Thus, the present disclosure provides a method of diagnosis and/or prognosis of a subject, comprising isolating one or more proteins (preferably, protein kinases) from a sample taken from the subject by contacting the sample with the compound and/or solid support disclosed herein. Such methods may comprise subsequent steps of determining the presence and/or level of expression of one or more specific proteins in the sample, and determining the diagnosis and/or prognosis for the subject based on the presence and/or level of expression of the one or more specific proteins in the sample.

Thus, the methods disclosed herein may be used to diagnose the presence of a disease or a predisposition to a disease in a subject, the method comprising:

detecting the presence of one or more proteins in a sample taken from the subject by contacting the sample with the compound or solid support disclosed herein,

wherein the presence of the one or more proteins in the sample is indicative of the disease or predisposition thereto.

In addition, the methods disclosed herein may be used to monitor a subject's response to a therapeutic treatment for a disease, the method comprising:

detecting the presence of one or more proteins in a sample taken from the subject by contacting the sample with the compound or solid support disclosed herein at a first time point; and

detecting the presence of one or more proteins in a sample taken from the subject by contacting the sample with the compound or solid support disclosed herein at a second, later time point after the subject has been exposed to a therapeutic treatment,

wherein the presence of the one or more proteins in the sample at the second time point is indicative of the subject's response to the therapeutic treatment.

The methods may further comprise determining the level of expression of one or more specific proteins in the sample. The methods may further comprise determining the diagnosis and/or prognosis for the subject and/or the subject's response to a therapeutic treatment based on the presence and/or level of expression of the one or more specific proteins in the sample.

The one or more proteins detected in the sample may be one or more protein kinases. Specific protein kinases may be associated with particular diseases or conditions and the presence (or level of expression) of such specific protein kinases may therefore be indicative of a particular disease or condition, as will be appreciated by a person skilled in the art.

The type of disease is not limiting on the application of the methods disclosed herein. Thus, the methods disclosed herein can be performed to diagnose the presence of any disease or predisposition thereto in a subject, or to monitor a subject's response to a therapeutic treatment for any disease.

In one example, the disease is cancer. As used herein, the term “cancer” shall be taken to include a disease that is characterized by uncontrolled growth of cells within a subject. The term “cancer” shall not be limited to cancer of a specific tissue or cell type. Those skilled in the art will be aware that as a cancer progresses, metastases occur in organs and tissues outside the site of the primary cancer. For example, in the case of many cancers, metastases commonly appear in a tissue selected from the group consisting of lymph nodes, lung, breast, liver, kidney and/or bone. Accordingly, the term “cancer” as used herein shall be taken to include a metastasis of a cancer in addition to a primary tumour. Exemplary cancers include breast cancer, ovarian cancer, colon cancer, head and neck cancer, lung cancer, pancreatic cancer and/or prostate cancer.

In another example, the disease is an inflammatory condition. Inflammatory conditions are a class of conditions characterized by movement of leukocytes (e.g., granulocytes) to a localized position in a subject's body, e.g., in a tissue. Inflammatory conditions can be chronic or acute. Exemplary inflammatory conditions include (but are not limited to) autoimmune diseases including insulin-dependent diabetes mellitus (or type 1 diabetes), insulin autoimmune syndrome, rheumatoid arthritis, psoriatic arthritis, chronic lyme arthritis, lupus, multiple sclerosis, inflammatory bowel disease including Crohn's disease, ulcerative colitis, celiac disease, autoimmune thyroid disease, autoimmune myocarditis, autoimmune hepatitis, pemphigus, anti-tubular basement membrane disease (kidney), familial dilated cardiomyopathy, Goodpasture's syndrome, Sjogren's syndrome, myasthenia gravis, polyendocrine failure, vitiligo, peripheral neuropathy, autoimmnune polyglandular syndrome type I, acute glomerulonephritis, adult-onset idiopathic hypoparathyroidism (AOIH), alopecia totalis, Hashimoto's thyroiditis, Graves' disease, Addison's disease, chronic beryllium syndrome, ankylosing spondylitis, juvenile dermatomyositis, polychondritis, scleroderma, regional enteritis, distal ileitis, granulomatous enteritis, regional ileitis, and terminal ileitis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barr syndrome, hemochromatosis, Henoch-Schonlein purpura, idiopathic IgA nephropathy, insulin autoimmune syndrome, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, inflammatory bowel disease, osteoarthritis, thyroiditis, and others. The term “inflammatory condition” also includes (but is not limited to) inflammation associated with diseases including acne vulgaris, asthma, chronic prostatitis, pancreatitis, glomerulonephritis, hypersensitivities, inflammatory bowel diseases, pelvic inflammatory disease, reperfusion injury, sarcoidosis, transplant rejection, vasculitis, interstitial cystitis, myopathy, cancer, or atherosclerosis.

In another example, the disease is a cardiovascular disease. Exemplary cardiovascular diseases include (but are not limited to) ischaemic heart disease (IHD), angina pectoris, coronary heart disease, stroke, transient ischaemic attacks, cerebrovascular disease, hypertensive disease, aortic aneurysm, peripheral arterial disease, retinal arterial disease and others.

In another example, the disease is a neurological disorder or a neurodegenerative disease. Exemplary neurological disorders or neurodegenerative diseases include (but are not limited to) Parkinson's Disease, Alzheimer's Disease, dementia with Lewy bodies, Huntington's disease, amyotrophic lateral sclerosis, multiple sclerosis, prion diseases, and others known in the art.

In a particular example, the disease is an inflammatory condition (for example, rheumatoid arthritis, inflammatory bowel disease, or other inflammatory condition), a cardiovascular disease, a neurological disorder or neurodegenerative disease, type II diabetes, or autosomal dominant polycystic kidney disease.

The present disclosure also provides a method of screening for an agent capable of binding a protein, the method comprising contacting the compound disclosed herein with a sample comprising one or more proteins in the presence and in the absence of a test agent, and identifying the test agent as an agent capable of binding a protein if the level of binding of the compound to the one or more protein kinases present in the sample is reduced in the presence, compared to the absence of the test agent. Preferably, the protein is a protein kinase. The sample may be any of the samples described herein. In one embodiment, the sample is a cell sample. Thus, the present disclosure provides a method of identifying novel binding targets for putative or known protein-binding agents (such as therapeutic agents) in a cellular environment, for investigating the binding specificity of putative or known protein-binding therapeutics, for investigating the binding specificity of other, known protein kinase inhibitors, and other uses.

### Kits

The present disclosure also provides a kit comprising the compound disclosed herein and/or the solid support disclosed herein and instructions for use. The instructions may define particularly preferred reaction conditions for performing any of the methods defined herein. In one example, the kit may further comprise one or more reagents suitable for carrying out an ELISA assay, for example, an antibody array.

Specific embodiments of the invention will now be described with reference to the following, non-limiting examples.

### EXAMPLES

### Experimental Procedures

Unless otherwise stated the following generalisations apply.

¹H NMR spectra were recorded on a Bruker Ultrashield plus (400 MHz) spectrometer. The multiplicity of a signal is designated by one of the following abbreviations: s, singlet; d, doublet; t, triplet; q, quartet; br, broad; m, multiplet. All observed coupling constants, J, are reported in Hertz. ¹³C NMR spectra were recorded on a Bruker Ultrashield plus (101 MHz) spectrometer in a broad band decoupled mode.

LC/MS data was generated using an Agilent 6100 Series Single Quad LC/MS having the following specifications:

Instrument: Agilent 6100 Series Single Quad LC/MS

Agilent 1200 Series HPLC

Pump: 1200 Series G1311A Quaternary pump

Autosampler: 1200 Series G1329A Thermostatted Autosampler

Detector: 1200 Series G1314B Variable Wavelength Detector

Liquid chromatography (LC) conditions:

Reverse Phase HPLC analysis

Column: Luna C8(2) 5u 50×4.6 mm 100 A

Column temperature: 30° C.

Injection Volume: 5 uL

Solvent A: Water 0.1% Formic Acid

Solvent B: Acetonitrile 0.1% Formic Acid

Gradient: 5-100% B over 10 min

Detection: 254 nm or 214 nm

Mass Spectometry (MS) conditions:

Ion Source Quadrupole

Ion Mode Multimode-ES

Drying gas temp: 300° C.

Vaporizer temperature: 200° C.

Capillary voltage (V): 2000 (positive)

Capillary voltage (V): 4000 (negative)

Scan Range: 100-1000

Step size: 0.1 sec

Acquisition time: 10 min

Analytical thin-layer chromatography was performed on Merck silica gel 60F254 aluminium-backed plates which were visualised using fluorescence quenching under UV light or acidic anisaldehyde or a basic potassium permanganate dip. Flash chromatography was performed using a Biotage Isolera purification system using either Grace or Biotage silica cartridges.

Where necessary anhydrous solvents were purchased from Sigma-Aldrich.

### Example 1

### Synthesis of 2-((5-Chloro-2-((4-(piperazin-1-yl)phenyl)amino)pyrimidin-4-yl)amino)-N-methylbenzamide (1) (CTx-0294885)

CTx-0294885 was prepared according to the following reaction scheme:

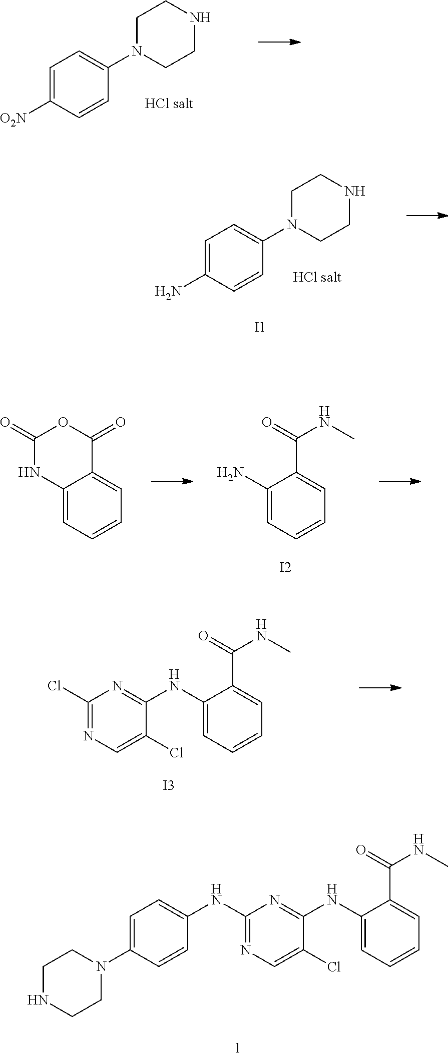

### (a) 4-(piperazin-1-yl)aniline hydrochloride (I1)

A suspension of 1-(4-nitrophenyl)piperazine hydrochloride (2.00 g, 8.21 mmol) and Pd/C (200 mg) in ethanol (50 mL) was stirred at room temperature under a hydrogen atmosphere for 72 hours. The resulting mixture was diluted with ethanol (100 mL), filtered through celite, washing the celite with 96% ethanol (2×100 mL). The combined filtrates were evaporated and the residue dried under vacuum to give the title compound (I1) (858 mg, 59%) as a tan solid; ¹H NMR (400 MHz, d₆-DMSO) δ 9.05 (br s, 1H), 6.76-6.68 (m, 2H), 6.56-6.47 (m, 2H), 4.92 (br s, 2H), 3.19-3.13 (m, 4H), 3.13-3.08 (m, 4H). LCMS: rt 0.96 min; m/z 178.2 [M+H].

### (b) 2-Amino-N-methylbenzamide (I2)

Methylamine hydrochloride (3.10 g, 46.0 mmol), ethanol (50 mL) and triethylamine (6.41 mL, 46.0 mmol) were stirred at room temperature for five minutes then isatoic anhydride (5.00 g, 30.7 mmol) was added. The mixture was heated at reflux for two hours under nitrogen and then allowed to cool to ambient temperature. The resulting mixture was concentrated, and the residue suspended in water (300 mL). The aqueous mixture was extracted with ethyl acetate (3×200 mL) then the combined ethyl acetate phases were washed with brine, dried (sodium sulphate) and evaporated. Chromatography (40 g silica cartridge, 0-80% ethyl acetate in petroleum benzine 40-60° C.) gave the title compound (I2) (3.97 g, 86% yield) as a pale pink solid; ¹H NMR (400 MHz, CDCl₃) δ 7.29 (dd, J=7.9, 1.5 Hz, 1H), 7.19 (ddd, J=8.4, 7.2, 1.5 Hz, 1H), 6.67 (dd, J=8.2, 1.1 Hz, 1H), 6.63 (ddd, J=8.2, 7.3, 1.2 Hz, 1H), 6.11 (s, 1H), 5.46 (s, 2H), 2.95 (d, J=4.8 Hz, 3H); ¹³C NMR (101 MHz, CDCl₃) δ 170.14, 148.61, 132.22, 127.23, 117.34, 116.68, 116.38, 26.56. LCMS: rt 2.61 min, m/z 120.2 [M-NHMe]⁺.

### (c) 2-((2,5-Dichloropyrimidin-4-yl)amino)-N-methylbenzamide (I3)

2-Amino-N-methylbenzamide (I2) (1.97 g, 13.1 mmol) was dissolved in isopropanol (40 mL) then DIPEA (2.28 mL, 13.1 mmol) and 2,4,5-trichloropyrimidine (1.25 mL, 10.9 mmol) were added and the mixture heated at reflux. After 5 hours the resulting mixture was cooled to room temperature, filtered and the collected solid washed with isopropanol (2×10 mL). The resulting solid was air dried to give the title compound (I3) (2.80 g, 86% yield) as a white solid; ¹H NMR (400 MHz, d₆-DMSO) δ 8.85 (d, J=5.3 Hz, 1H), 8.52 (dd, J=8.5, 1.2 Hz, 1H), 8.46 (s, 1H), 7.80 (dd, J=7.9, 1.6 Hz, 1H), 7.59 (ddd, J=8.7, 7.4, 1.6 Hz, 1H), 7.22 (td, J=8.0, 1.2 Hz, 1H), 2.81 (d, J=4.5 Hz, 3H); ¹³C NMR (101 MHz, d₆-DMSO) δ 168.69, 156.62, 156.14, 155.27, 138.27, 131.82, 128.10, 123.10, 121.04, 120.81, 114.91, 26.33. LCMS: rt 5.73 min; m/z 297.0, 299.0 [M+H]⁺, 266.0, 268.0 [M-NHMe]⁺.

### (d) 2-((5-Chloro-2-((4-(piperazin-1-yl)phenyl)amino)pyrimidin-4-yl)amino)-N-methylbenzamide (1) (CTx-0294885)

2-((2,5-Dichloropyrimidin-4-yl)amino)-N-methylbenzamide (I3) (149 mg, 0.503 mmol), 4-(piperazin-1-yl)aniline hydrochloride (I1) (134 mg, 0.627 mmol), 2,2,2-trifluoroethanol (5 mL) and concentrated aqueous HCl (2 drops) were stirred at 95° C. for 16 hours. On cooling to ambient temperature the resulting mixture was diluted with 5% aqueous sodium hydroxide (100 mL) then extracted with ethyl acetate (3×100 mL). The combined ethyl acetate phases were washed with brine, dried (sodium sulfate) and evaporated. Chromatography (12 g C18 cartridge, 0-40% acetonitrile/methanol) gave a residue which was washed with dichloromethane (2×2 mL) then dried to give the title compound (I) (33.4 mg, 15% yield) as an off white solid; ¹H NMR (400 MHz, d₆-DMSO) δ 9.19 (s, 1H), 8.78-8.71 (m, 2H), 8.15 (s, 1H), 7.74 (dd, J=7.8, 1.3 Hz, 1H), 7.49-7.42 (m, 3H), 7.12 (t, J=7.2 Hz, 1H), 6.86 (d, J=9.0 Hz, 2H), 3.00-2.95 (m, 4H), 2.86-2.79 (m, 7H). LCMS: rt 4.28 min; m/z 438.1 [M+H]⁺.

### Example 2

### Conjugation of 2-((5-Chloro-2-((4-(piperazin-1-yl)phenyl)amino)pyrimidin-4-yl)amino)-N-methylbenzamide (1) (CTx-0294885) to a Solid Support

2-((5-chloro-2-((4-(piperazin-1-yl)phenyl)amino)pyrimidin-4-yl)amino)-N-methylbenzamide (1) (CTx-0294885) was immobilised onto activated CH-Sepharose® 4B resin according to the following reaction:

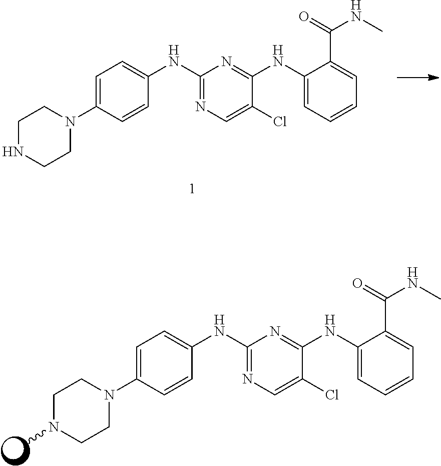

Activated CH-sepharose® 4B (1.78 g) was swelled with 1 mM aqueous HCl (50 mL), and collected by filtration (porosity 4 glass frit) then washed with additional 1 mM aqueous HCl (9×50 mL). 2-((5-Chloro-2-((4-(piperazin-1-yl)phenyl)amino)pyrimidin-4-yl)amino)-N-methylbenzamide (1) (14 mg, 32 mol) was dissolved in DMF (5.3 mL), and diluted with 100 mM sodium bicarbonate (5.3 mL). The resin was added and the resulting suspension agitated on a shaker table for 18 hours. The mixture was filtered and the resin washed with 50% aqueous DMF (2×15 mL). The resin was then suspended in 1 M ethanolamine in 50% aqueous DMF (10 mL), and agitated for one hour. The resulting mixture was filtered, and the resin washed sequentially with 50% aqueous DMF (10×10 mL), 0.1 M pH 4 sodium acetate buffer (20×25 mL), 0.1 M pH 8 sodium bicarbonate buffer (20×25 mL) and 20% aqueous ethanol (10×20 mL). The collected resin was suspended in 20% aqueous ethanol and stored at 4° C. By LCMS analysis of pre and post-coupling reagent solutions, >99% of amine (1) was immobilised on the resin.

### Example 3

### Characterization of Proteins Bound by 2-((5-Chloro-2-((4-(piperazin-1-yl)phenyl)amino)pyrimidin-4-yl)amino)-N-methylbenzamide (1) (CTx-0294885)

The following general experimental procedures were adhered to in the following examples.

### Cell Culture and Cell Lysis

MDA-MB-231 cells were cultured in RPMI1640 (Invitrogen) supplemented with 10% fetal bovine serum (Invitrogen) and insulin at 0.25 IU/ml. Subconfluent cells were lysed with ice-cold lysis buffer containing 50 mM HEPES-NaOH (pH 7.5), 150 mM NaCl, 0.5% Triton X-100, 1 mM EDTA 1 mM EGTA supplemented with additives (10 μg/ml aprotinin, 10 μg/ml leupeptin, 1 mM PMSF, 10 mM NaF, 50 ng/ml calyculin A, 1% phosphatase inhibitor mixture 3 (Sigma), and 2.5 mM Na₃VO₄) for 5 min on ice. Cell debris was removed by centrifugation at 16,500 g at 4° C. for 30 min and the supernatant was subsequently filtered through a 0.45 μm PVDF membrane (Millipore). Protein concentrations were measured by the Bradford assay (Bio-Rad).

### Generation of Kinase Affinity Resin

Kinase inhibitors Purvalanol B (Tocris) and SU6668 (Biochempartner Chemical) were immobilized to EAH-sepharose 4B (GE Healthcare) and VI16832 (Evotec) was coupled to ECH-sepharose 4B (GE Healthcare) beads via a carbodiimide-mediated reaction. Briefly, 1 ml of beads were washed 3 times with 10 ml of 0.5 M NaCl, twice with 10 ml H₂O and once with coupling buffer made of 50% dimethylformamide (DMF) and 50% ethanol. The washed and aspirated beads were then mixed with 1 ml of inhibitor solution (Purvalanol B at 10 mM, SU6668 at 10 mM and VI16832 at 3 mM dissolved in coupling buffer), followed by dropwise addition of 150 μl of 1 M N-(3-dimethylaminopropyl)-N-ethylcarbodiimide hydrochloride (EDC) in coupling buffer.

The coupling reaction was carried out in the dark at room temperature overnight on a rotating wheel. To block the remaining reactive groups, the resin was washed twice with 2 ml of coupling buffer followed by addition of 1 ml of DMF/ethanol/ethanolamine at ratio 1:1:1 (pH 8). The reaction was initiated by dropwise addition of 150 μl of 1 M EDC in coupling buffer and the mixture was then incubated again in the dark at room temperature overnight with gentle agitation. Subsequently the resin was washed 3 times with 10 ml of coupling buffer, twice with 10 ml of 0.5 M NaCl and once with 10 ml of 20% ethanol. The resin was stored in 20% ethanol as a suspension at 4° C. in the dark. For CTx-0294885 immobilization, activated CH Sepharose 4B (GE Healthcare) (0.5 g) was swelled with 20 ml of 1 mM HCl and collected by vacuum filtration. The collected resin was washed 4 times with 20 ml of 1 mM HCl, and mixed with 3 ml of CTx-0294885 (9 mM) dissolved in 50% DMF 50% 100 mM sodium bicarbonate (pH 8). The suspension was agitated on a shaker table for 18 h. The mixture was washed with 5 ml of 50% aqueous DMF and the resin was resuspended in IM ethanolamine in 50% aqueous DMF. The mixture was agitated for 1 h before the resin was collected by filtration and washed 5 times with 5 ml of 50% aqueous DMF, 10 times with 10 ml of 0.1 M acetate buffer (pH 4), 10 times with 10 ml of 0.1 M bicarbonate buffer (pH 8), and 10 times of 10 ml 20% ethanol. The resin was resuspended in 10 ml of 20% ethanol and stored at 4° C. An equally suitable alternative protocol for CTx-0294885 immobilization is described in Example 2.

### Kinase Enrichment

The salt concentration in the protein lysates was adjusted to 1 M NaCl and the kinase inhibitor resins were washed once with 10 ml of H₂O and once with 10 ml of washing buffer A (lysis buffer with 1 M NaCl plus 10 mM NaF and 0.1 mM Na₃VO₄) prior to kinase enrichment. For testing of single kinase inhibitors, 1 ml of inhibitor resin was incubated with 50 mg of protein lysates, and for multi-inhibitor resins, a cocktail comprising 1 ml of each inhibitor resin was used for incubation with 100 mg of protein lysates. After 2 h of incubation at 4° C. in the dark on a rotating wheel, the resins were washed twice with 10 ml of washing buffer A, once with 10 ml of washing buffer B (same as washing buffer A except NaCl concentration is 150 mM instead of 1 M) and once with washing buffer C (50 mM HEPES, 10 mM NaF and 0.1 mM Na₃VO₄). Resin-bound proteins were eluted using 4 ml 5 mM dithiothreitol (DTT), 0.5% SDS, with 3 min incubation at 60° C. for 5 consecutive rounds. The pooled elution fractions were then lyophilized and resuspended in 3 ml of H₂O followed by acetone precipitation of protein.

### Acetone Precipitation

The protein sample was mixed with at least 8 volumes of ice-cold acetone, briefly vortexed before being incubated at −20° C. for 2 h. Proteins were pelleted by centrifugation at 13,000 g at 4° C. for 10 min and the resulting protein pellet was washed twice with 70% ethanol before re-dissolving in Laemmli sample buffer or 20 mM HEPES buffer (pH 7.5) containing 8 M urea depending on downstream applications.

Gel Electrophoresis and in-Gel Digestion with Trypsin

Seventy percent of the precipitated proteins was dissolved in 2× Laemmli sample buffer with incubation at 90° C. for 5 min before separation on a 10% SDS-PAGE gel at 100 V for 90 min. The gel was fixed with 10% methanol, 7.5% acetic acid for 30 min, and stained until protein bands became visible with in-house prepared Coomassie blue solution (0.186% Coomassie brilliant blue G-250, 24.8% methanol, 2.28% phosphoric acid and 12.5% ammonium sulphate). The gel was cut into 12 pieces and destained twice using 50% acetonitrile (ACN) in 50 mM NH₄HCO₃ (pH 8.5) for 20 min. Gel pieces were washed twice with equilibration buffer (50 mM NH₄HCO₃) and vacuum dried prior to protein reduction with 25 mM DTT for 30 min and alkylation with 55 mM iodoacetamide (IAA) for 30 min. Gel pieces were washed twice with equilibration buffer for 5 min followed by dehydration with 100% ACN and vacuum centrifugation. Proteins were enzymatically digested at 37° C. overnight with modified sequencing grade trypsin (Promega) at a trypsin to protein ratio of 1:50. The tryptic digestion reaction was stopped by addition of 5% formic acid (FA) and the resulting peptides were extracted by incubation and agitation in equilibration buffer containing 50% ACN followed by 100% ACN. The peptide mixtures were vacuum dried and acidified with H₂O containing 0.2% trifluoroacetic acid (TFA) for desalting using in-house made C₁₈ StageTips.

### In-Solution Digestion

The remaining 30% of the precipitated protein sample was solubilized in 20 mM HEPES buffer (pH 7.5) containing 8 M urea and then reduced, alkylated and digested with modified trypsin as described in the previous section. The digestion reaction was stopped by acidifying the sample to pH<2.5 with 100% TFA and the resulting peptides were subsequently purified using C₁₈ StageTips.

### Desalting Using C₁₈ StageTips

The C₁₈ StageTip was made in-house according to a published protocol (Rappsilber, 2007). After activation of the C₁₈ StageTip with 20 μl of methanol and equilibration with 20 μl 10.1% TFA, the TFA acidified peptide sample (pH<2.5) was gently forced through the C₁₈ StageTip column with a syringe. The column was washed 3 times with 20 μl of 0.1% TFA. Purified peptides were eluted using 30 μl of 0.1% TFA, 80% ACN, and the eluted fraction was concentrated in a speedy-vac to a final volume of 2-3

### Phosphopeptide Enrichment Using TiO₂

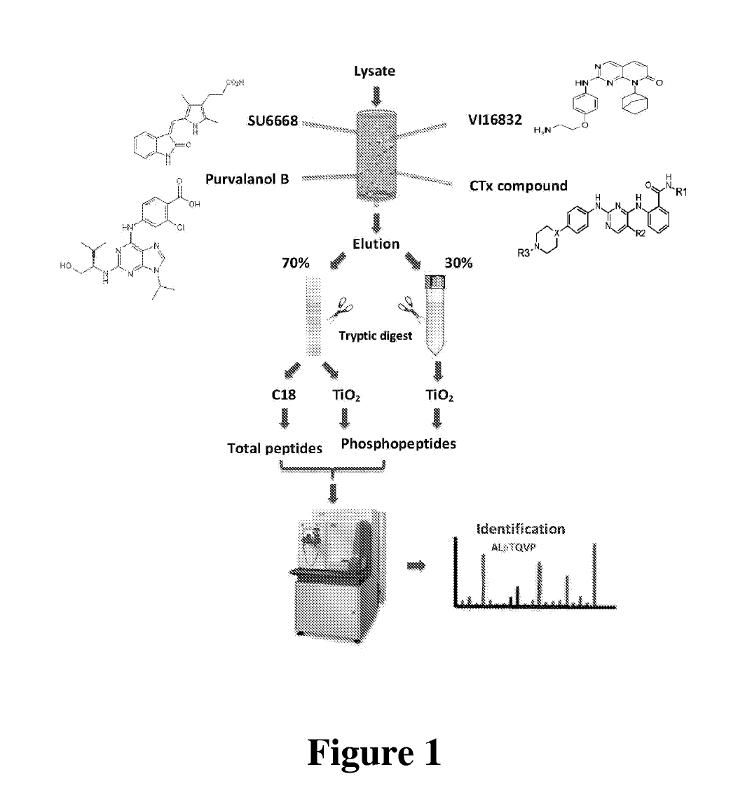

All purified peptides from the in-solution digests and 90% of the purified peptides of each fraction extracted from the in-gel digests were subjected to phosphopeptide enrichment using titanium dioxide beads (GL Sciences) (FIG. 1). Trypsin digests from adjacent gel slices were combined to give a total of 6 peptide samples. TiO₂ beads were suspended in 100% ACN and packed onto a C₈ disc in the home-made C8 StageTip by centrifugation at 2000 g for 2 min. Peptide samples dissolved in loading buffer (25% lactic acid, 73% ACN and 2% formic acid) were added separately onto the packed TiO₂ columns followed by centrifugation of the column at 1000 g for 10 min to allow the peptide solution to slowly pass through the column. The flow-through from each column was then re-applied to a new TiO₂ column until 2 or 5 rounds of consecutive phosphopeptide enrichment were performed for in-solution digests and in-gel digests respectively. The columns were washed 4 times with 50 μl of washing buffer (1% TFA, 80% ACN) prior to phosphopeptide elution using 50 μl of 5% ammonia solution in MilliQ H₂O, and a subsequent second elution with 50 μl of 30% ACN. The two elution fractions were combined, freeze-dried and cleaned up using a C₁₈ StageTip. Samples were stored at −20° C. until mass spectrometry analysis.

### LC-MS/MS Data Acquisition

Digest peptides were separated by nano-LC using an Ultimate 3000 HPLC and autosampler system (Dionex). Samples were concentrated and desalted onto a micro C₁₈ precolumn (500 μm×2 mm, Michrom Bioresources) with H₂O:CH₃CN (98:2, 0.05% TFA) at 15 μl/min. After a 4 min wash the pre-column was switched (Valco 10 port valve, Dionex) into line with a fritless nano column (75μ×˜10 cm) containing C18 media (5μ, 200 Å Magic, Michrom). Peptides were eluted using a linear gradient of H₂O:CH₃CN (98:2, 0.1% formic acid) to H₂O:CH₃CN (64:36, 0.1% formic acid) at 250 nl/min over 30 min. High voltage 2000 V was applied to low volume tee (Upchurch Scientific) and the column tip positioned ˜0.5 cm from the heated capillary (T=280° C.) of an Orbitrap Velos (Thermo Electron) mass spectrometer. Positive ions were generated by electrospray and the Orbitrap operated in data dependent acquisition mode (DDA).

A survey scan m/z 350-1750 was acquired in the Orbitrap (Resolution=30,000 at m/z 400, with an accumulation target value of 1,000,000 ions) with lockmass enabled. Up to the 15 most abundant ions (>5,000 counts) with charge states>+2 were sequentially isolated and fragmented within the linear ion trap using collisionally induced dissociation with an activation q=0.25 and activation time of 30 ms at a target value of 30,000 ions. m/z ratios selected for MS/MS were dynamically excluded for 30 s.

### Protein Identification and Data Analysis

Raw files generated by the mass spectrometer were processed with the MaxQuant software (version 1.1.1.28) and the extracted peak lists were searched against the UniProtKB/Swiss-Prot Homo sapiens database (Version_{—}2010_{—}10; including common contaminants) and a decoy database. The search parameter was selected as follows: cystein carbamidomethylation was set as fixed modification; methionine oxidation, protein N-acetylation, phosphorylation of serine, threonine and tyrosine were selected as variable modification; minimum required peptide length was 6 and up to 2 missed cleavages were allowed; the initial mass tolerance was 20 ppm for precursor ions and 0.5 Da for fragment ions; match between run was selected; the false discovery rate was 1% for both protein and peptide identifications. Peptides with posterior error probability greater than 10% as well as proteins without a unique peptide were filtered out in the downstream analysis. For phosphopeptide analysis, phosphorylation sites were assigned by MaxQuant and only sites with a localization probability >0.75 were considered as correctly assigned. For enrichment analysis of gene ontology categories, non-protein kinases bound by CTx-0294885 from 2 replicate experiments were submitted to the DAVID Bioinformatic database online (http://david.abcc.ncifcrf.gov/, version 6.7) to compare with a reference dataset comprising of all UniProt entries and their respective GO identifiers (Huang et al., 2009a, 2009b).

### Results

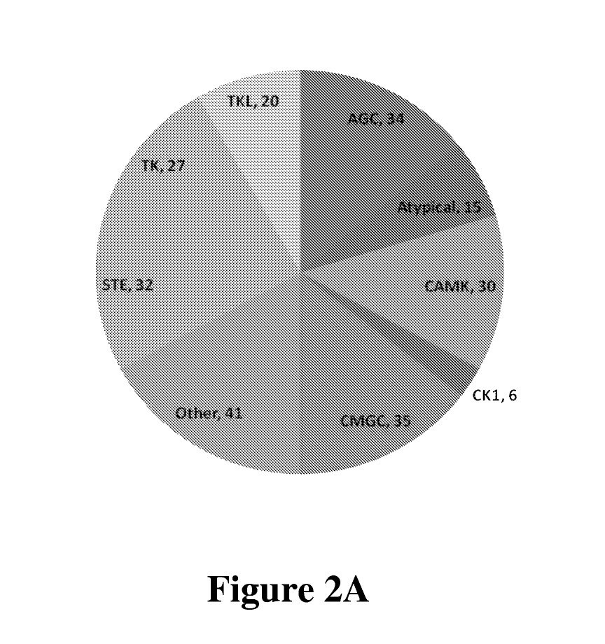

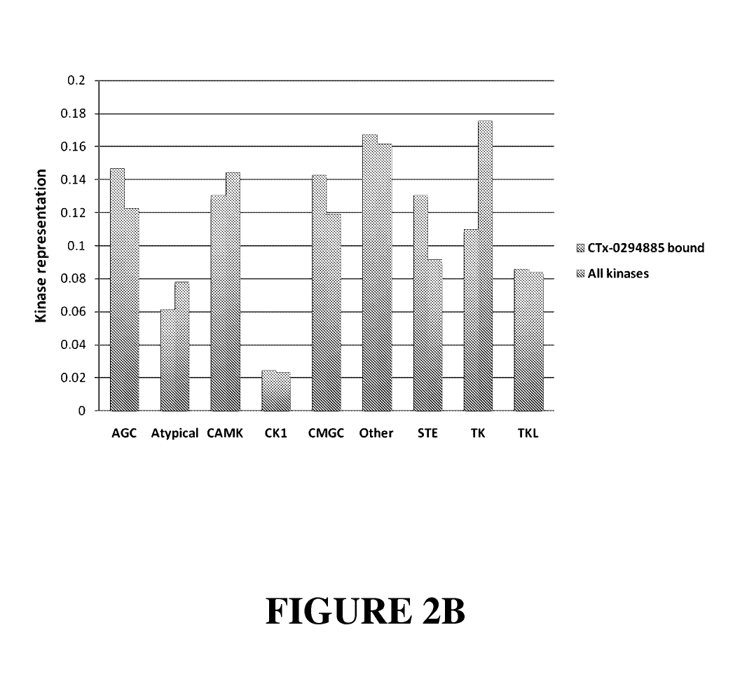

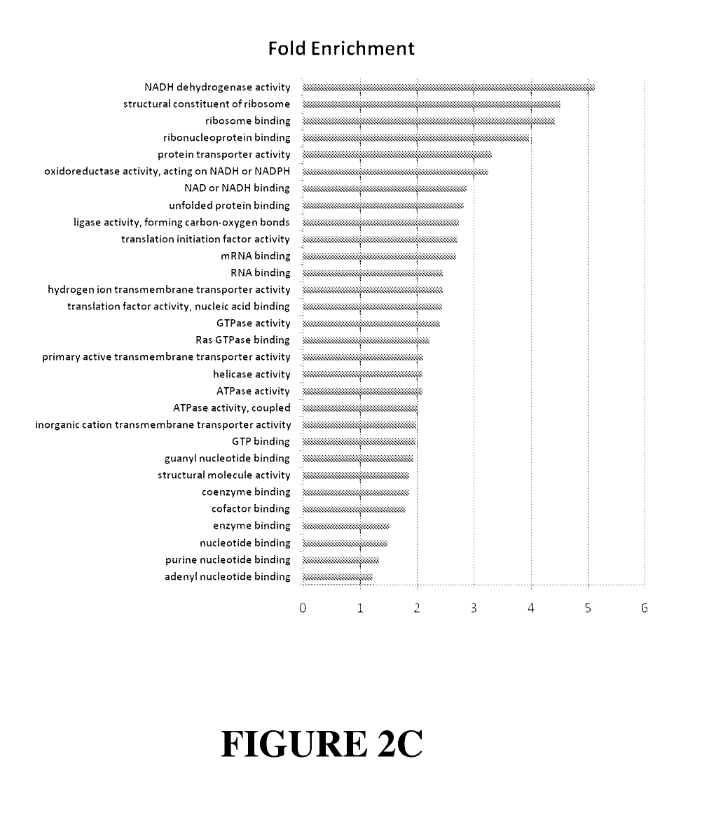

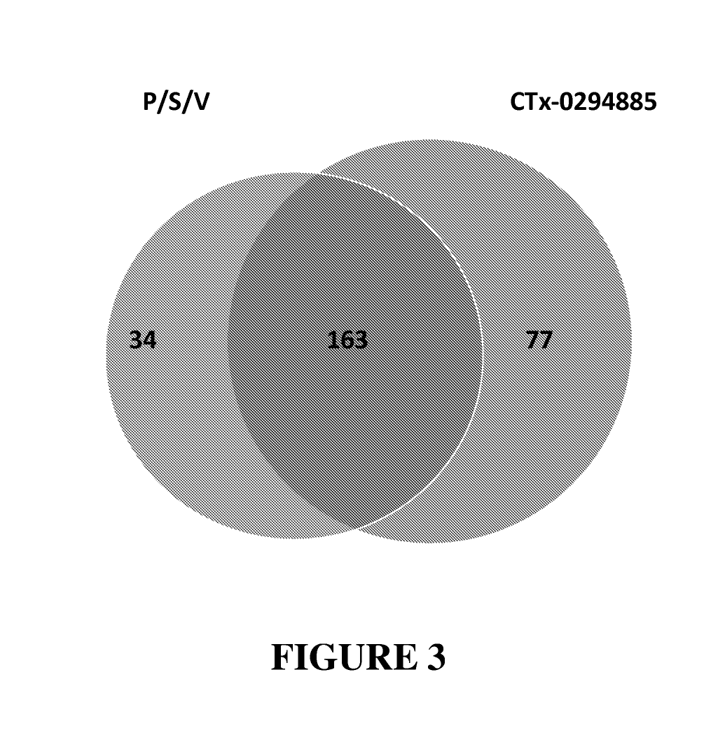

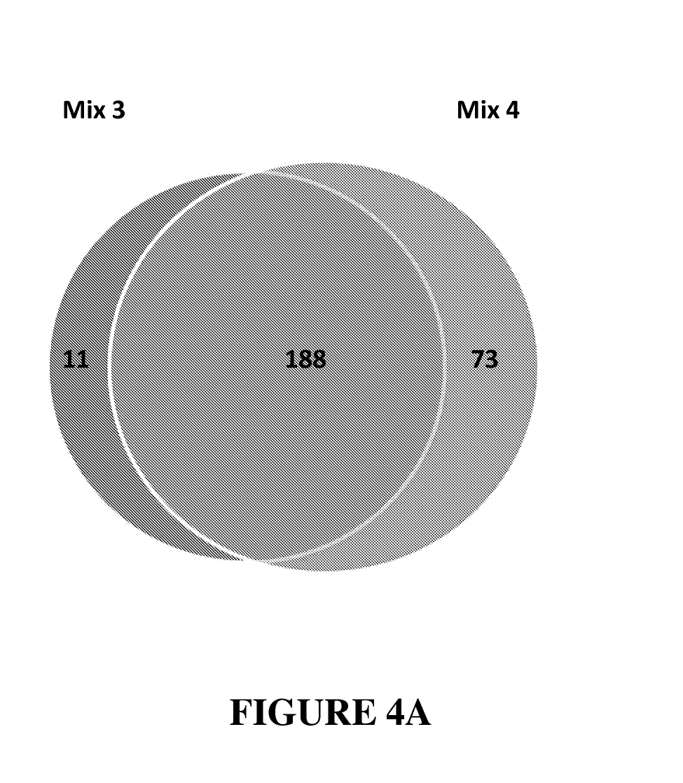

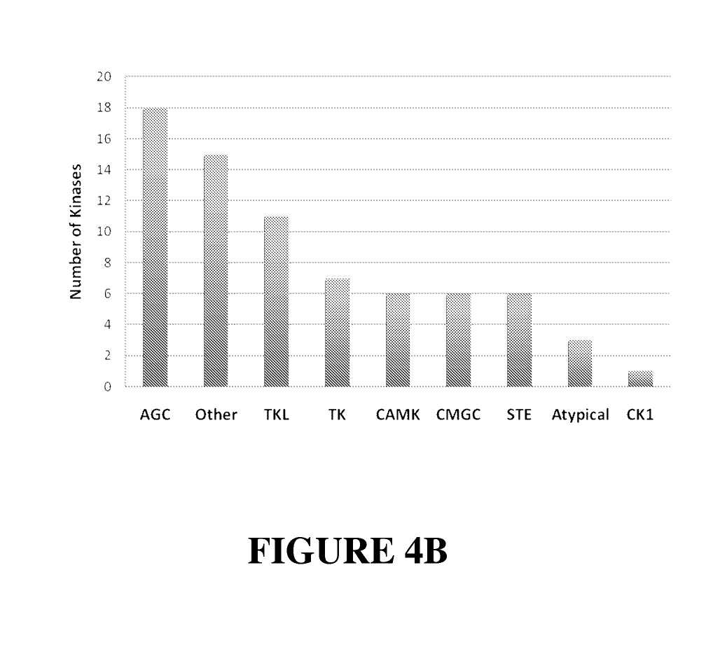

In the purification of proteins from MDA-MB-231 breast cancer cells using CTx-0294885 coupled to CL sepharose 4B beads, 2546 proteins were identified, 185 of which were kinases. If phosphosites are included, then this increases to 240 kinase identifications. Reproducible results were obtained from two independent biological replicates. Representatives from all of the 9 major kinase subgroups were identified, indicating broad coverage of the expressed kinome (FIG. 2A). The compound gave particularly strong enrichment for kinases of the STE, CMGC and AGC subfamilies (FIG. 2B). CTx-0294885-mediated purification also led to enrichment for other purine nucleotide binding proteins. Gene ontology analysis of non-protein kinases purified by CTx-0294885 indicated enrichment for a variety of terms within the ‘molecular function’ category, including oxidoreductase activity, GTPase activity and purine nucleotide binding (FIG. 2C). Of note, these also represent ‘druggable targets’.

The binding selectivity of CTx-0294885 was compared with that of 3 other commonly used kinase-capture reagents: purvalanol B (P), SU6668 (S) and VI16832 (V). Combining the individual totals for kinases bound by P, S and V gave 197 kinases, while CTx-0294885 alone bound 240. CTx-0294885 bound 77 kinases not bound by the other reagents (FIG. 3).

This suggested that combining CTx-0294885 with the other reagents to make a combined affinity matrix should greatly extend kinome coverage (a mammalian cell expresses ˜300 protein kinases). This was confirmed. An affinity matrix (Mix 4) that combined CTx-0294885 with P+S+V (Mix 3) purified ˜2600 proteins, 261 of which were kinases. 806 phosphorylation sites were identified on 183 kinases. In addition, a direct comparison of Mix 4 and Mix 3 revealed that the former purifies 73 additional kinases, with a notable increase in the number of AGC kinases detected (FIGS. 4A and 4B).

Thus CTx-0294885 represents an effective kinase capture reagent that can be used either alone, or in combination with other reagents, to purify protein kinases (and other purine nucleotide binding proteins) from cell or tissue samples. Other compounds falling within the scope of claim 1 have been shown to retain the same function of CTx-0294885 and can therefore be used for the same purposes. For example, a compound of Formula I wherein R1=H, R2=Cl, X=N and R3=COCH₃ mimics the immobilised form of CTx-0294885 and retains the same function of CTx-0294885. Applications of the kinase capture reagent of the present disclosure include: profiling kinase expression and/or activation in different cell lines, tissue specimens or disease states, leading to the identification of potential therapeutic targets and diagnostic/prognostic biomarkers; use as a component of a diagnostic or prognostic test that requires pre-fractionation of protein kinases; use in competition assays for selectivity screening of other kinase inhibitors; use in assays that monitor the effects of kinase inhibitors on specific signalling pathways and complexes; and other uses.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Manning et al., Science, 2002. 298(5600): p. 1912-34.
Su et al., Proc Natl Acad Sci USA, 2002. 99(7): p. 4465-70.
Nolen et al., Mol Cell, 2004. 15(5): p. 661-75.
Lemmon and Schlessinger, Cell, 2010. 141(7): p. 1117-34.
Knight et al., Nat Rev Cancer, 2010. 10(2): p. 130-7.
Brognard and Hunter, Curr Opin Genet Dev, 2011. 21(1): p. 4-11.
Pao and Chmielecki, Nat Rev Cancer, 2010. 10(11): p. 760-74.
Cohen, Nat Rev Drug Discov, 2002. 1(4): p. 309-15.
Belmonte and Blaxall, Circ Res, 2011. 109(3): p. 309-19.
Su and Tsai, Annu Rev Cell Dev Biol, 2010.
Donath and Shoelson, Nat Rev Immunol, 2011. 11(2): p. 98-107.
Qin et al., J Clin Invest, 2010. 120(10): p. 3617-28.
Macek et al., Annu Rev Pharmacol Toxicol, 2009. 49: p. 199-221.
Hochgrafe et al., Cancer Res, 2010. 70(22): p. 9391-401.
Daub et al., Mol Cell, 2008. 31(3): p. 438-48.
Bantscheff et al., Nat Biotechnol, 2007. 25(9): p. 1035-44.
Oppermann et al., Mol Cell Proteomics, 2009. 8(7): p. 1751-64.
Wissing et al., Mol Cell Proteomics, 2007. 6(3): p. 537-47.
Rappsilber et al., Nature Protocol, 2007. 2: p. 1896-906.
Huang et al., (a) Nature Protoc, 2009. 4(1): p. 44-57.
Huang et al. (b) Nucleic Acids Res, 2009. 37(1): p. 1-13.
Toker and Yoeli-Lerner, Cancer Res, 2006. 66(8): p. 3963-3966.
Zdychova and Komers, Physiol Res, 2005. 54: p. 1-16.

1. A compound of the following general Formula I:
Formula I 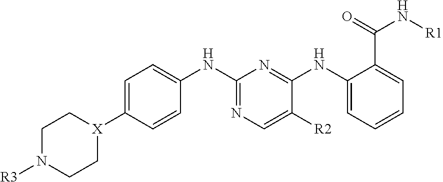
wherein R1=H or Me; R2=CF₃ or Cl; R3=H, COCH₃ or a linker group; and
X=N or C.

2. The compound of claim 1, wherein R1=H, R2=Cl, X=N and R3=H, COCH₃ or a linker group.

3. The compound of claim 1 or claim 2, wherein R1=H, R2=Cl, X=N and R3=H.

4. The compound of any preceding claim, attached to a solid support.

5. The compound of claim 4, wherein the solid support comprises a plurality of sepharose beads.

6. A method of synthesizing the compound of any preceding claim, the method comprising reacting a compound of the following formula F4 (wherein R1=H or Me; X=N or C; and the CF₃ group can optionally be substituted with Cl) 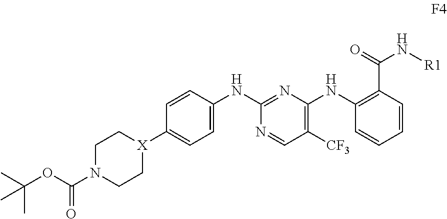
with an acid, such as trifluoroacetic acid, under suitable reaction conditions.

7. A method of synthesizing the compound of any one of claims 1-5, the method comprising reacting an amino pyridine of the formula F6 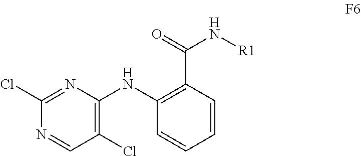
with an aniline of formula 11 or formula 12 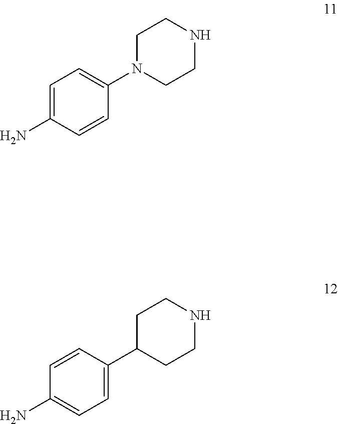 [SMILES: Nc1ccc(N2CCNCC2)cc1 ; Nc1ccc(C2CCNCC2)cc1]
under suitable reaction conditions.

8. A solid support comprising the compound of any one of claims 1-3 attached thereto.

9. The solid support of claim 8, which comprises a plurality of sepharose beads.

10. The solid support of any one of claims 8-9, further comprising an additional binding agent attached thereto.

11. The solid support of claim 10, wherein the additional binding agent is any one or more of bis(III) indoyl-maleimide, purvalanol B, staurosporine, CZC8004, sunitinib, vandetanib, VI16832, bisindoylmaleimide X, AX14596 and SU6668.

12. The solid support of claim 11, wherein the additional binding agent is any one or more of purvalanol B, VI16832 and SU6668.

13. A method of detecting the presence of one or more proteins in a sample, the method comprising contacting the sample with the compound of any one of claims 1-5 or the solid support of any one of claims 8-12.

14. The method of claim 13, wherein the one or more proteins is a protein kinase.

15. The method of claim 14, wherein the protein kinase is a protein kinase of the STE, CMGC or AGC subfamily of protein kinases, or of the Akt family of kinases.

16. The method of any one of claims 13-15, wherein the protein is present in a sample taken from a subject.

17. The method of claim 16, wherein the sample comprises a cell taken from a subject.

18. The method of claim 17, wherein the cell is lysed before contacting the sample with the compound of any one of claims 1-5 or the solid support of any one of claims 8-12

19. The method of any one of claims 16-18, wherein the sample is taken from a subject suffering from or suspected of suffering from a disease.

20. The method of claim 19, wherein the disease is cancer or an inflammatory condition.

21. A method of diagnosing the presence of a disease or a predisposition to a disease in a subject, the method comprising:
detecting the presence of one or more proteins in a sample taken from the subject by contacting the sample with the compound of any one of claims 1-5 or the solid support of any one of claims 8-12,
wherein the presence of the one or more proteins in the sample is indicative of the disease or predisposition thereto.

22. A method of monitoring a subject's response to a therapeutic treatment for a disease, the method comprising:
detecting the presence of one or more proteins in a sample taken from the subject by contacting the sample with the compound of any one of claims 1-5 or the solid support of any one of claims 8-12 at a first time point; and
detecting the presence of one or more proteins in a sample taken from the subject by contacting the sample with the compound of any one of claims 1-5 or the solid support of any one of claims 8-12 at a second, later time point after the subject has been exposed to a therapeutic treatment,
wherein the presence of the one or more proteins in the sample at the second time point is indicative of the subject's response to the therapeutic treatment.

23. The method of claim 21 or claim 22, further comprising determining the level of expression of one or more specific proteins in the sample.

24. A method of isolating a protein from a sample, the method comprising contacting the sample with the compound of any one of claims 1-5 or the solid support of any one of claims 8-12.

25. A method of screening for an agent capable of binding a protein kinase, the method comprising:
contacting the binding agent of any one of claims 1-5 or the solid support of any one of claims 8-12 with a sample comprising one or more protein kinases in the presence and in the absence of a test agent, and
identifying the test agent as an agent capable of binding a protein kinase if the level of binding of the binding agent of any one of claims 1-5 or the solid support of any one of claims 8-12 to the one or more protein kinases present in the sample is reduced in the presence, compared to the absence of the test agent.

26. A kit comprising the compound of any one of claims 1-5 and/or the solid support of any one of claims 8-12 and instructions for use.
